(19) United States
(12) Patent Application Publication
(10) Pub. No.: US 2026/0151259 A1
(43) Pub. Date: Jun. 4, 2026

(54) ICE COMPRESS COMPONENT AND ICE COMPRESS PROTECTIVE CLOTHING

(71) Applicant: Wuhan Pakcare Bio-Technology Co.,Ltd., Wuhan (CN)

(72) Inventors: Yao QIN, Wuhan (CN); Shouwei XU, Wuhan (CN)

(73) Assignee: Wuhan Pakcare Bio-Technology Co.,Ltd., Wuhan (CN)

(21) Appl. No.: 19/420,795

(22) Filed: Dec. 16, 2025

(30) Foreign Application Priority Data
Nov. 30, 2024 (CN) 2024229415346

(51) Int. Cl. A61F 7/02; A41D 13/005

(52) U.S. Cl. CPC A61F 7/02; A41D 13/0053; A61F 2007/0268

(57) ABSTRACT

The present disclosure discloses an ice compress component and an ice compress protective clothing. The ice compress component includes a heat insulation layer, a cold storage layer, an ice compress layer and a skin-friendly layer arranged sequentially and all made of flexible materials. The cold storage layer and the skin-friendly layer are in close fit with the two opposite sides of the ice compress layer respectively, the cold storage layer is provided with a plurality of independent ice grids, each of the plurality of ice grids is provided with a cold insulation agent inside, and the ice compress layer is made of gel. By means of the dual ice compress effect achieved by combining the cold storage layer and the ice compress layer, along with the heat insulation effect of the heat insulation layer, the duration of a single ice compress application is greatly increased.

### CROSS-REFERENCE TO RELATED APPLICATIONS

The application claims priority to Chinese patent application No. 2024229415346, filed on Nov. 30, 2024, the entire contents of which are incorporated herein by reference.

### TECHNICAL FIELD

The present disclosure relates to the technical field of ice compress, and in particular to an ice compress component and an ice compress protective clothing.

### BACKGROUND

Currently, sports elbow braces, patellar braces, and other protective clothing products on the sports market are mainly designed to provide support and stability for the elbows and patella of the knee joints. The conventional protective clothing is mostly made of a single elastic material. During exercise, the heat generated by the body is trapped by the tightly fitting strap material, and the large amount of heat accumulated in a local area cannot be effectively released, which will cause discomfort to the body. An ice compress protective clothing is developed for this purpose. While numerous ice compress protective clothing exist in the current technology, they mostly focus on variations in appearance and usage, failing to address several core issues in ice compress therapy: 1. the product itself has a too short duration of low temperature, which cannot meet the needs of long-term ice compress and does not achieve the expected therapeutic effect; 2. after freezing, the product becomes stiff and does not fit the applied area well, resulting in poor using effect; and 3. the surface temperature of the ice compress product is uneven, which easily leads to localized low temperatures and the risk of frostbite. Therefore, an ice compress component and an ice compress protective clothing are provided to solve the above problems.

### SUMMARY

One of the objectives of the present disclosure is to provide an ice compress component and an ice compress protective clothing to solve the problem that existing an ice compress protective clothing has a short duration of ice compress.

The ice compress component and ice compress protective clothing of the present disclosure are capable of being realized through the following technical solutions:

An ice compress component of the present disclosure includes a heat insulation layer, a cold storage layer, an ice compress layer and a skin-friendly layer arranged sequentially and all made of flexible materials. The cold storage layer and the skin-friendly layer are in close fit with the two opposite sides of the ice compress layer respectively, the cold storage layer is provided with a plurality of independent ice grids, each of the plurality of ice grids is provided with a cold insulation agent inside, and the ice compress layer is made of gel.

In one embodiment, the heat insulation layer is made of nylon or polyester combined with foamed cotton and fiber cotton.

In one embodiment, the plurality of ice grids are distributed in a matrix.

In one embodiment, the cold storage layer is made of a co-extruded film of PE, PVC, TPU or nylon.

In one embodiment, the ice compress layer is made of gel made of polyacrylamide, glycerin, sodium alginate and gelatin.

In one embodiment, the skin-friendly layer is made of knitted fabric made of polyester, nylon, spandex, cotton, modal or bamboo fiber.

An ice compress protective clothing of the present disclosure includes the ice compress component as described in any one of items, and a protective clothing component, where the ice compress component is arranged on the protective clothing component.

In one embodiment, the ice compress component is detachably arranged on the protective clothing component.

In one embodiment, the protective clothing component includes a protective clothing body, where the ice compress component is arranged on the protective clothing body; and a fixing belt with one end fixedly connected to the protective clothing body, where the ice compress component is fixedly attached to a part needing ice compress by the fixing belt.

In one embodiment, a hook surface of a hook-and-loop fastener and a loop surface of the hook-and-loop fastener are arranged on the two opposite sides of the fixing belt respectively, the hook surface of the hook-and-loop fastener is arranged at the end of the fixing belt away from the protective clothing body, the loop surface of the hook-and-loop fastener is in a long strip shape, and the hook surface of the hook-and-loop fastener and the loop surface of the hook-and-loop fastener are detachably bonded.

Compared with the prior art, the ice compress component and the ice compress protective clothing of the present disclosure have the following beneficial effects:

According to the ice compress component and the ice compress protective clothing of the present disclosure, by means of the dual ice compress effect achieved by combining the cold storage layer and the ice compress layer, along with the heat insulation effect of the heat insulation layer, the duration of a single ice compress application is greatly increased, and the problem of a short duration of ice compress of an existing ice compress protective clothing is effectively solved. The ice compress layer is made of gel, which retains high elasticity and ultra-softness even after freezing, so as to be in close fit with the part needing ice compress during ice compress, which is combined with the flexible-bending ice grids of the cold storage layer and the skin-friendly fabric of the innermost layer, thereby greatly improving the fit degree between the product and the ice-compressed part and the use comfort. Meanwhile, the plurality of ice grids in the cold storage layer are isolated from the body surface by the gel, so that the ice grids are incapable of making direct contact with the skin surface of the ice-compressed part, and are only responsible for continuously delivering cooling energy to the gel. Furthermore, the ice grids in independent chambers are evenly distributed in size, which is capable of achieving a balanced ice compress effect, thereby reducing the risk of localized frostbite and greatly improving the using safety of the ice compress product.

### BRIEF DESCRIPTION OF DRAWINGS

To more clearly illustrate the technical solutions of the embodiments of the present disclosure, the accompanying drawings used in the embodiments will be briefly introduced below. It should be understood that the following drawings only show some embodiments of the present disclosure and should not be regarded as a limitation on the scope. For those skilled in the art, other related drawings can be obtained based on these drawings without creative effort.

FIG. 1 is a schematic structural diagram of an ice compress component of the present disclosure;

FIG. 2 is a schematic diagram of an exposed structure of the ice compress component of the present disclosure shown in FIG. 1, including a cold storage layer;

FIG. 3 is a schematic structural diagram of the cold storage layer shown in FIG. 2;

FIG. 4 is a schematic structural diagram of an ice compress protective clothing of the present disclosure in use; and

FIG. 5 is a schematic structural diagram of the ice compress protective clothing of the present disclosure as shown in FIG. 4 in an unfolded state.

Reference numerals: 10—Ice compress component; 11—Heat insulation layer; 12—Cold storage layer; 121—Ice grid; 13—Ice compress layer; 14—Skin-friendly layer; 20—Protective clothing component; 21—Protective clothing body; 22—Fixing belt; 221—Loop surface of hook-and-loop fastener; 222—Hook surface of hook-and-loop fastener.

### DETAILED DESCRIPTION OF THE EMBODIMENTS

To make the objectives, technical solutions, and advantages of the embodiments of the present disclosure clearer, the technical solutions of the embodiments of the present disclosure will be clearly and completely described below with reference to the accompanying drawings. Obviously, the described embodiments are only some embodiments of the present disclosure, and not all embodiments. The mechanisms of the embodiments of the present disclosure described and illustrated in the accompanying drawings can typically be arranged and designed in a variety of different configurations.

Therefore, the following detailed description of embodiments of the present disclosure provided in the accompanying drawings is not intended to limit the scope of the claimed disclosure, but merely to illustrate selected embodiments of the present disclosure. Based on the embodiments of the present disclosure, all other embodiments obtained by those skilled in the art without creative effort are within the scope of protection of the present disclosure.

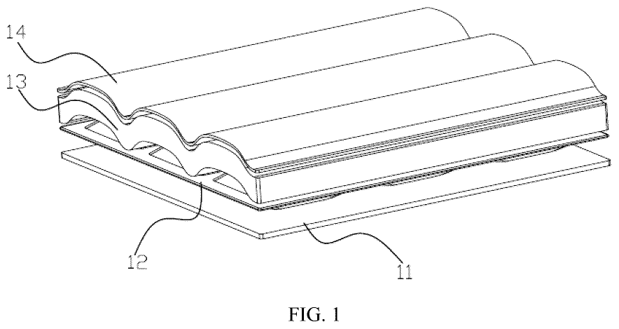

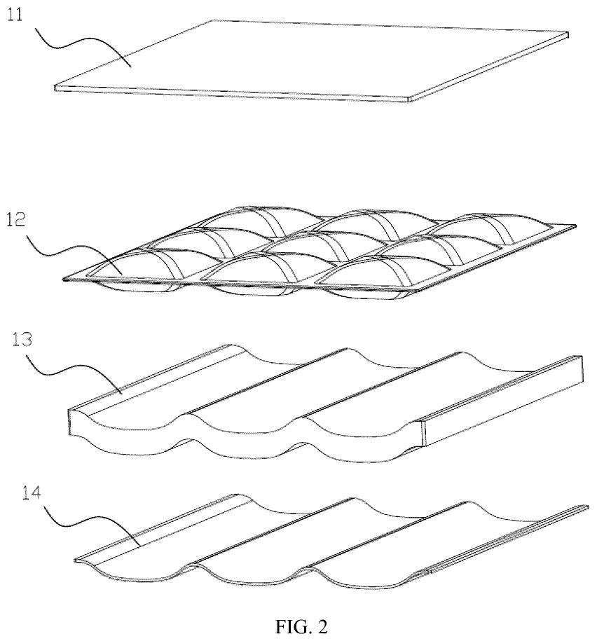

Please refer to FIGS. 1 and 2, an ice compress component 10 of the present disclosure mainly includes a heat insulation layer 11, a cold storage layer 12, an ice compress layer 13 and a skin-friendly layer 14 arranged sequentially and all made of flexible materials to match ice compress operation of different parts. The heat insulation layer 11 has a heat insulation effect, reduces the conduction of low temperature from the cold storage layer 12 to the outside through the heat insulation layer 11, thereby enabling the cold storage layer 12 to maintain a low temperature effect for a long period of time. The cold storage layer 12 is in close fit with the ice compress layer 13, and conducts low temperature to the ice compress layer 13, thereby greatly prolonging the duration of low temperature of the ice compress layer 13. The skin-friendly layer 14 is in close fit with the other side of the ice compress layer 13, and is in close contact with the part needing ice compress, which greatly improves the fit between the ice compress component 10 and the ice-compressed part and the use comfort.

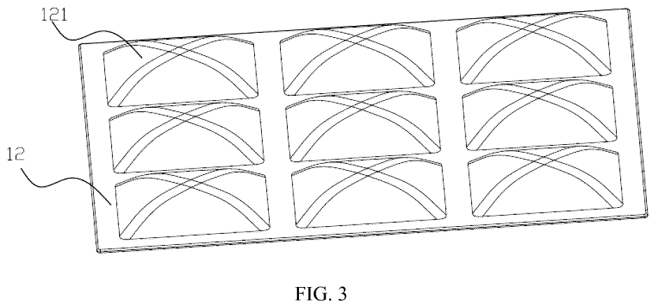

Please refer to FIGS. 1-3, in this embodiment, the heat insulation layer 11 is made of nylon or polyester combined with foam cotton and fiber cotton, and has the characteristics of good heat insulation effect and soft texture. Specifically, the cold storage layer 12 is provided with a plurality of independent ice grids 121, the plurality of ice grids 121 are arranged in a matrix and respectively provided with a cold insulation agent inside. Each of the plurality of ice grids 121 is capable of being flexibly bent after freezing, so that the ice compress component 10 better adapts to different cold-compressed parts more flexibly, and meanwhile continuously transmits cold energy to the ice compress layer 13, which greatly prolonging the duration of ice compress and enhancing the ice compress effect. The cold storage layer 12 is made of a co-extruded film of PE, PVC, TPU or nylon, thereby having the characteristics of high strength and soft texture.

Please refer to FIGS. 1 and 2, specifically, the ice compress layer 13 is made of a gel made of polyacrylamide, glycerin, sodium alginate and gelatin, which has the characteristics of high elasticity, super flexibility and high water retention. Even after freezing, the gel is still capable of maintaining high elasticity and super flexibility, and is capable of changing the shape of itself according to the different shapes of the ice compress component during ice compress, so that the ice compress component 10 is in fit with the surface layer of the ice compress component more flexibly and closely, thereby achieving a better ice compress effect. The skin-friendly layer 14 is made of knitted fabric made of polyester, nylon, spandex, cotton, modal or bamboo fiber, which has the characteristics of super elasticity, super softy and high hygroscopicity, ensuring a soft, comfortable and healthy skin-friendly feel during use.

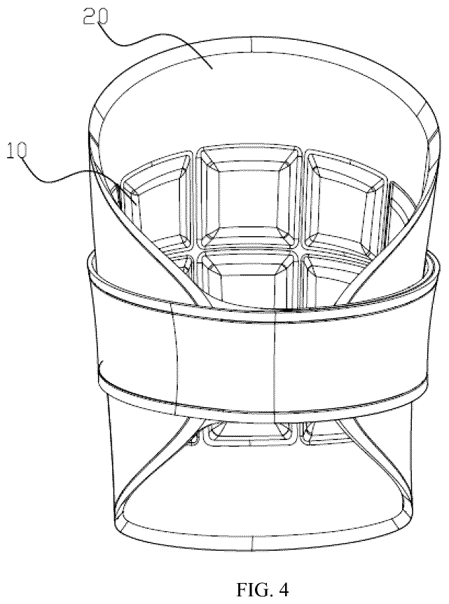

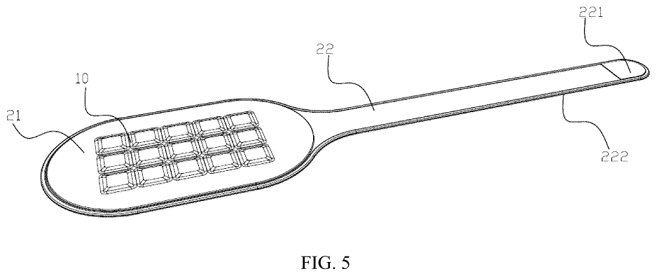

Please refer to FIGS. 4 and 5, an ice compress protective clothing of the present disclosure includes the ice compress component 10 and a protective clothing component 20. The ice compress component 10 is arranged on the protective clothing component 20. Preferably, the ice compress component 10 is detachably arranged on the protective clothing component 20. The protective clothing component 20 is detachably arranged on the part needing ice compress. In this embodiment, the ice compress protective clothing 20 includes a protective clothing body 21 and a fixing belt 22. The ice compress component 10 is arranged on the protective clothing body 21. One end of the fixing belt 22 is fixedly connected to the protective clothing body 21, and the ice compress component 10 is fixedly attached to a part needing ice compress by the fixing belt 22. Specifically, a hook surface 221 of a hook-and-loop fastener and a loop surface 222 of the hook-and-loop fastener are arranged on the two opposite sides of the fixing belt 22 respectively, the hook surface 221 of the hook-and-loop fastener is arranged at the end of the fixing belt 22 away from the protective clothing body 21, the loop surface 222 of the hook-and-loop fastener is in a long strip shape, and the hook surface 221 of the hook-and-loop fastener and the loop surface 222 of the hook-and-loop fastener are detachably bonded. By means of the cooperation of the two, the ice compress component 10 is fixed to the part needing ice compress.

It should be noted that the specific usage process of the ice compress component and ice compress protective clothing of the present disclosure is as follows: the frozen ice compress component 10 is attached to the part needing ice compress. Due to the flexibility characteristic, the ice compress component 10 is in close fit with the part needing ice compress, and then is fixed by the fixing belt 22.

The technical features of the above embodiments can be combined in any way. For the sake of brevity, not all possible combinations of the technical features in the embodiments are described. However, as long as there is no contradiction in the combination of these technical features, they should be considered to be within the scope of this specification.

The above embodiments are merely examples of several implementation methods of the present disclosure, and their descriptions are relatively specific and detailed, but they should not be construed as limiting the scope of the patent. It should be noted that those skilled in the art can make various modifications and improvements without departing from the concept of the present disclosure, and these modifications and improvements are all within the scope of protection of this invention. Therefore, the scope of protection of this invention patent shall be determined by the appended claims.

What is claimed is:

1. An ice compress component, comprising a heat insulation layer, a cold storage layer, an ice compress layer and a skin-friendly layer arranged sequentially and all made of flexible materials, wherein the cold storage layer and the skin-friendly layer are in close fit with the two opposite sides of the ice compress layer respectively, the cold storage layer is provided with a plurality of independent ice grids, each of the plurality of ice grids is provided with a cold insulation agent inside, and the ice compress layer is made of gel.

2. The ice compress component according to claim 1, wherein the heat insulation layer is made of nylon or polyester combined with foamed cotton and fiber cotton.

3. The ice compress component according to claim 1, wherein the plurality of ice grids are distributed in a matrix.

4. The ice compress component according to claim 1, wherein the cold storage layer is made of a co-extruded film of PE, PVC, TPU or nylon.

5. The ice compress component according to claim 1, wherein the skin-friendly layer is made of knitted fabric made of polyester, nylon, spandex, cotton, modal or bamboo fiber.

6. An ice compress protective clothing, comprising the ice compress component as described in claim 1, and a protective clothing component, wherein the ice compress component is arranged on the protective clothing component.

7. The ice compress protective clothing according to claim 6, wherein the ice compress component is detachably arranged on the protective clothing component.

8. The ice compress protective clothing according to claim 6, wherein the protective clothing component comprises a protective clothing body, wherein the ice compress component is arranged on the protective clothing body; and a fixing belt with one end fixedly connected to the protective clothing body, wherein the ice compress component is fixedly attached to a part needing ice compress by the fixing belt.

9. The ice compress protective clothing according to claim 8, wherein a hook surface of a hook-and-loop fastener and a loop surface of the hook-and-loop fastener are arranged on the two opposite sides of the fixing belt respectively, the hook surface of the hook-and-loop fastener is arranged at the end of the fixing belt away from the protective clothing body, the loop surface of the hook-and-loop fastener is in a long strip shape, and the hook surface of the hook-and-loop fastener and the loop surface of the hook-and-loop fastener are detachably bonded.
